(19) United States
(12) Patent Application Publication
(10) Pub. No.: US 2007/0060906 A1
(43) Pub. Date: Mar. 15, 2007

(54) Method, reagent, and device for embolizing blood vessels in tumors with ultrasonic radiation micro-bubble reagent

(76) Inventor: Wei Wu, Nanjing (CN)

Correspondence Address: Kirton & McConkie, 1800 Eagle Gate Tower, 60 East South Temple, Salt Lake City, UT, 84145-0120, US

(21) Appl. No.: 10/556,237

(22) PCT Filed: May 28, 2004
(86) PCT No.: PCT/CN04/00555

§ 371 Date: Nov. 13, 2006

(30) Foreign Application Priority Data
Aug. 18, 2003 (CN) 03132373.1
Sep. 1, 2003 (CN) 03152895.3
Sep. 23, 2003 (CN) 03158291.5
Feb. 20, 2004 (CN) 200410014108.2
Mar. 19, 2004 (CN) 200410014367.5

(51) Int. Cl. A61M 31/00; A61H 1/00; A61B 17/20

(52) U.S. Cl. 604/507; 601/50; 604/22

(57) ABSTRACT

A tracing or marking isotope micro-bubble reagent with targeting substance, the combination or mixture of ultrasonic micro-bubble imaging reagent with tracing or marking isotope. The tracing or marking isotope includes generally medical used isotopes. The micro-bubble reagent includes fluoro-carbon type, medical salt water type, semi lactose bubble liquid type, envelope bubble liquid type and carbon dioxide generating type. Large molecule substance is selected as the carrier of ultrasonic reagent to wrap, stick, stabilize and carry the bubbles. The tracing or marking isotope micro-bubble reagent with targeting substance is applied to position the tumor area in the treatment of tumor by using low frequency, low energy ultrasonic to induce the cavitation effect of micro-bubbles to form thrombus embolus in blood vessels, Meanwhile, radial β isotope is applied to radiate the tumor cells by using the ionizing radiation biological effect thereof.

### 1. TECHNICAL FIELD

This Invention relates to the method, reagent and device for embolizing blood vessels in tumors with an ultrasonic radiation micro-bubble reagent. This invention includes the application of an ultrasonic radiation micro-bubble reagent to form thrombus embolus in blood vessels, especially the application of the ultrasonic radiation micro-bubble reagent to form thrombus embolus in blood vessels and subsequently limit or eliminate tumors.

2. BACKGROUND

Malignant tumors are common and endanger human health. Statistics from the Chinese Ministry of Hygiene show that cancer is prevalent in China. There are about 2 million new cases and about 1.5 million deaths each year.

More than one hundred years ago our ancestors discovered that there are more blood vessels in tumor tissue than in normal tissues. In 1971, J. D. Folkman of Harvard Medical School hypothesized that the tumor growth depends on blood vessels, and this theory has been gaining acceptance. In recent years, medical experts in China and other countries have found that tumor vasculature is the morphologic basis for the growth and spread of tumor cells. Blood vessels of tumors not only supply nutrients to tumor cells but also spread tumor cells to other parts of the human body. Therefore, the interdiction of the blood supply and restriction of the formation of neo-vasculature have become a new and effective way to treat tumors.

Currently, surgery is still the preferred treatment for early stage tumors but with significant risks of tumor metastasis and recurrence. Chemotherapy, radiotherapy and thermotherapy are applied to kill tumor cells. All of the above treatments can eliminate or restrict the growth of new blood vessels of tumors, but they can not interdict or control the formation of new vessels. These treatments cause severe poisoning and other harmful side effects. The existing tumor embolizing treatment can only be used for medium size or larger artery and vein due to the limitations of tools and surgical methods. The treatment is not able to treat the micro blood vessel networks which supply blood directly to the tumor tissue. In summary, although the research on the theory of neo-vasculature in tumors has taken a big step forward, the treatment still faces many difficulties.

Ultrasonic technology has been used for clinical diagnosis since the 1940s. The rapid development of medicine, physics, etc. and their intercrossing have brought about new uses of ultrasound technology many areas of medicine. The use ultrasonic technology has penetrated the health care, disease prevention and treatment (including extracorporeal shock wave lithotripsy (ESWL), hemostasis, tumor removal, etc.), biotechnology and other medical fields. Ultrasonic biology research revealed the interactive mechanism of ultrasonic radiation with every hiberarchy of biological system. The cavitation effect is one of the most important effects of ultrasonic biology. The cavitation effect is generated by ultrasonic radiation on cavitation nuclide. The degree of the cavitation effect is related to the concentration of the cavitation nuclides. Since the quantity of cavitation nuclides in mammals is very small, high power ultrasonic radiation is necessary to generate a cavitation effect. This high power radiation causes severe damage to the biological tissues. Chinese and international research indicate that high power ultrasonic radiation (higher intensity focus ultrasonic, HIFU) can induce the cavitation of nuclides in tissues to generate a cavitation effect. The targeted tissue is crushed by high pressure and temperature, but its treatment area can not be precisely controlled (such as HIFU) so it can not be utilized for blood vessel embolizing treatment of tumors.

In recent years, the research of ultrasonic contrast reagent in ultrasonic imaging diagnosis is very active. But the research of using micro-bubbles to strengthen the ultrasonic biological effects for treatment is still in the early stages. Ultrasonic radiation micro-bubble reagent continues to use the terminology of X-ray contrast imaging reagent in radiology and is called ultrasonic contrast reagent in medical ultrasound.

Since J. D. Folkman's famous theory, the treatment of targeting blood vessels (i.e. by embolizing blood vessels and interdicting the newly formed blood vessel to starve the tumor) has become a new treatment of tumors after surgery, chemotherapy and radiotherapy. This new treatment has become the first option for treating tumors which can not be removed by surgery or for the tumors which may recur after surgery.

Currently, the application of natural blood vessel forming promoter and inner-borne blood vessel forming inhibitor has become a new method to treat cancer. Interferon α-2α has been successfully used to treat lung blood vessel tumors which threaten children's lives. This treatment can even be used to treat blood vessel tumors which severely damages the tissues. Many blood vessel forming inhibitors have been used in clinical trials, such as using reorganized blood platelet factor 4 to treat terminal colon cancer, matrix metallo protein enzyme inhibitor to treat terminal cancer, kidney cell carcinoma, ovarian cancer and non small cell lung cancer. TNP470 has been approved by the FDA and has been used in clinical trials. Recently, Pfizer has developed a new type of anti-cancer medicine which targets the blood vessels formation in lung cancer and it has completed phase I clinical trials. The results showed significant anti-cancer effect when it is used together with chemotherapy. Most methods of anti-blood forming treatment are still at the experimental or clinical trial stages and have many problems. For instance, the cancer cells develop resistance to the anti-blood forming medicine. After the treatment is stopped, the tumors begin to grow again. An additional negative side effect is that it interdicts the concrescence of the wound. A comprehensive evaluation of this treatment should be made through long periods of clinical surveillance. Currently, anti-blood vessel forming treatment is at the preliminary and trial stages and thus difficult to assess its clinical application value.

Currently AVASTIN is the only genetically engineered monoclonal antibody in the world. It is a new kind of anti-cancer medicine that can inhibit the formation of blood vessels. AVASTIN was approved by the FDA in Feb. 2004 and it is the first anti-cancer medicine that applies the “starving the tumor” technology.

Below is a comparison of current applied treatments and devices for fighting cancer.

| | | | | Application | |
|---|---|---|---|---|---|
| Item | Treatments | Advantages | Disadvantages | Scope | Costs |
| 1 | Surgery | Removes the | Application strictly | Removes | Varies |
| | | tumor | restrained | the tumor | |
| | | Can eradicate | Not applied for most | at a precise | |
| | | the tumor | patients, multi- | area | |
| | | | pathological changes. | Functions | |
| | | | Chemotherapy and | of the | |
| | | | radiotherapy are still | organ are | |
| | | | needed for most of the | not | |
| | | | patients after surgery. | effected. | |
| 2 | Chemotherapy | Can proceed at | Apparent inhibiting on | Can | High |
| | and | any time before | marrow | proceed at | |
| | Radiotherapy | or after surgery | Weakens the immune | any time | |
| | | Available for | system | before or | |
| | | patients where | Not suitable for weak | after | |
| | | surgery is not | patients | surgery. | |
| | | suitable or the | | Available | |
| | | tumor has | | for patients | |
| | | metastasis or | | not suitable | |
| | | recurrence. | | for surgery | |
| | | | | or the | |
| | | | | tumor has | |
| | | | | metastasis | |
| | | | | or | |
| | | | | recurrence. | |
| | | | | Part of | |
| | | | | malignant | |
| | | | | tumors. | |
| 3 | Immunology | Improve the | Currently at the research | Malignant | Very high |
| | therapy | patient's | stage for most kinds of | tumors | |
| | | immune system | treatments | | |
| | | Controls tumor | | | |
| | | growth | | | |
| 4 | Gene | Control tumor | Currently at research | Malignant | Very high |
| | Therapy | growth from | stage | tumors | |
| | | gene level | Effects under evaluation | | |
| 5 | Microwave | Stable heating | Low penetration | Not for | Relatively |
| | Heating | Effective for | Hard to measure the | deep | Low |
| | | tumors just | temperature | tumors, | |
| | | below the | Radiation protection | esophagus | |
| | | surface. | needed | colon, | |
| | | Large radiation | Difficult to adjust | cervix or | |
| | | source by using | Not so effective for deep | other | |
| | | multi-source | tumors | antrum | |
| | | microwave. | | tumors. | |
| | | Applied for in- | | | |
| | | antrum | | | |
| | | treatments. | | | |
| 6 | Radiation | Larger volume | Undersurface fat is prone | Shallow | Moderate |
| | Heating | can be heated | to damage. | tumors | |
| | | Much deeper or | Uneven intensity | | |
| | | larger tumors | distribution | | |
| | | can be heated by | Radiation protection | | |
| | | installing a | needed | | |
| | | cooling loop | Difficult to adjust | | |
| | | water bag. | Not effective for deep | | |
| | | | tumors | | |
| 7 | HIFU | Good | Not applicable for organs | Medium | Relatively |
| | Heating | penetration | with air in | size tumors | high |
| | | Applied for | Complicated operation | Deeper | |
| | | shallow and | Difficult to attain | tumors | |
| | | deep tumors | temperature | | |
| | | High killing | High cost | | |
| | | rate | | | |

Ultrasonic radiation micro-bubble reagent is a type of reagent used for ultrasonic detection. Its main application is cardiac muscle imaging. Cha Daogang, etc., The effect of Ultrasonic Radiation on the Stability of Sound Vibrating Micro Bubbles. Vol. 19, Pub. No. 5, 1999, Transaction of The First Military Medical University. The main factors which affect the imaging of the cardiac muscle are the concentration and size of the micro-bubbles. Cha reported the adoption of the 2×2×4 analytical method to analyze the effect of different ultrasonic radiation on the concentration and size of the micro-bubbles. In other words, it analyzed the independent effect and interaction of sound wave frequency, energy and radiation duration on the concentration and size of the micro-bubbles. The research provided a reference for choosing the appropriate ultrasonic radiation condition for the clinical venous cardiac muscle acoustics trial detection. The research revealed that that the more destruction to the micro-bubbles and smaller size of the micro-bubbles when more energy and the radiation is longer; the radiation frequency has no significant effect on the concentration of the micro-bubbles, but it affects the size of the micro-bubbles; higher the frequency, the smaller the bubbles. One of the currently known ultrasonic micro-bubbles reagent uses protein to wrap the micro-bubbles. Such micro-bubbles circulate through the lung and arrive at the left section of the heart to facilitate the imaging of the cardiac muscle. Another reagent is the fluorocarbon micro-bubble reagent, which is prepared by taking 10 ml 5% human albumin solution in a plastic injector and placing on a sound vibration device. In this process, fluorocarbon gas is added to the albumin solution at a steady rate. The diameter of the reagent micro-bubbles obtained from this method is between 2.0˜5.0 μm, 98% of the bubbles are less than 10 μm; the concentration of the micro-bubbles is (1˜2)×10¹² per liter. After injecting this micro-bubble into the veins, the cardiac muscle image appears on a common ultrasonic device. The diagnostic principle is that the intensity of resonance is directly proportional to 106 of the radius of the micro-bubbles. So the micro-bubbles with high concentration and larger size have a high intensity of resonance. The reason why ultrasound becomes the first choice in valve disease imaging technology is because it can provide information on the blood flow dynamics, structure, function, severity, probable causes and potential valve disease.

There are numerous kinds of ultrasonic radiation micro-bubble reagents, such as Albunex and Optison which are approved for clinical use by the United States FDA.

Fluorocarbon micro-bubble reagent, medical salt-water micro-bubble reagent and the following ultrasonic radiation micro-bubble reagents are examples:
1) Albunex (Molecular Biosystems Inc., USA)
2) fso69 (Molecular Biosystems Inc., USA)
3) SHU454 (Schering AG, Germany)
4) SHU508 (Schering AG, Germany)
5) QW3600 (Sonus Pharmaceuticals Cosla Mesa)

The present fluorocarbon micro-bubble reagents are prepared with a human albumin solution and by a sound vibration device. In this process, fluorocarbon gas is added to the Albumin solution at a steady rate. Medical salt-water micro-bubble reagent follows a similar preparation process.

The present ultrasonic radiation micro-bubble reagents include medical salt-water types as mentioned above and human serum albumin type. They have been used in clinical research. For example, LEVOVIST (Schering AG, Germany) uses large molecule fatty acid as the main component of ultrasonic reagent. The large molecule substance can wrap, stick, stabilize and carry the bubbles. It is widely used today. Some ultrasonic radiation micro-bubble reagents mainly carry air and fluorocarbon gas. The imported ultrasonic radiation micro-bubble reagent is very expensive and it may not be the best option for the purpose of this invention. The effect of adding gas into medical salt-water is not good enough. Fluorocarbon human serum albumin micro-bubble reagent is made from blood products and may cause allergies and contagious blood-borne diseases.

In recent years, research of the application of micro-bubble reagent in ultrasonic imaging diagnosis is very active. It is just getting started in the area of using micro-bubble reagent to enhance the ultrasonic biological treatment. Reports concerning the formation of embolus in blood vessels, especially the formation of capillary blood vessels using an ultrasonic radiation micro-bubble reagent has not been found.

The current method of radiation immunologized imaging includes introducing the mono clone antibody with isotope marking via a specific path into the body and combining in the body with the antigen of tumor cells at a specific orientation. After a period of time, the radioactive concentration at the position of the tumor accumulates to a certain level. Camera or SPECT is used to facilitate the planar or layer display. The display can indicate the size, position and extent of the tumor and the metastasis foci. The application of such method includes and determination of the nature and stage of the tumor.

The current radiation immunological therapy is: use a peculiar antibody as the carrier, introduce the isotope nuclide which irradiates β- or α particles to the position of the tumor antigen to facilitate the inner radiation treatment of the tumor. This treatment is mostly through venous inoculation but it can also be achieved by administering medicine to a precise area. The effect has been proven by extensive clinical trials.

The ultrasonic treatment head can not be used on the human body directly. For instance, an ultrasonic wave in the water bath can be applied for lithotripsy and other treatments. But this kind of treatment limits its application and is inconvenient. The treatment head will cause tissue damage if the power ultrasonic wave is in direct contact with human skin tissue.

### 3. CONTENT OF THIS INVENTION

The purposes of this invention includes 1) to provide the method for embolizing blood vessels in tumors with an ultrasonic radiation micro-bubble reagent; 2) particularly to provide a carbon dioxide type ultrasonic radiation micro-bubble reagent of carbon dioxide type to form embolus in blood vessels 3) it includes the application of an ultrasonic radiation micro-bubble reagent to form embolus in blood vessels.

Another purpose of this invention is to provide a medical device using an ultrasonic micro-bubble reagent to form embolus in the capillary blood vessels. More specifically a device to treat benign and malignant tumors by forming embolus in the capillary blood vessels in the targeted area, especially by forming blood vessel embolus at the specific spot or in a certain area. This device includes a medically applied power ultrasonic treatment head, particularly a hand held ultrasonic treatment head with a coupling and buffer protecting device to achieve harmless ultrasonic transmission of power. It is easy to be used for treatment.

The objective of this invention also includes providing a kind of isotope tracing or marking micro-bubble reagent with a targeting substance and its application. The isotope marking micro-bubble reagent is highly special and is applied for inner-corporeal diagnosis and treatment. Such micro-bubble reagent can be used in the assessment of detecting and positioning of tumors in the application of ultrasonic radiation micro-bubble reagent in the process of forming embolus in blood vessels. The reagent also solves the conflict between detecting and treatment in the process of treating of tumors.

The purposes of this invention also include improving the effectiveness of the treatment of malignant tumor by forming blood vessel embolus at a precise location or in a certain area. This invention provides the integrated method of treating tumors by using the combination of the targeting tracing substance or marking isotope with an ultrasonic radiation micro-bubble reagent to form capillary embolus.

The method of this invention embolizes blood vessels in tumors with an ultrasonic radiation micro-bubble reagent to form blood vessel embolus. The reagent is injected into the location or the area where an embolus needs to be formed and then irradiates it with ultrasonic waves, resulting in an embolus. There are no specific requirements for the ultrasonic waves but low energy and low frequency waves are preferred. Ultrasonic waves themselves cause no harmful side effects to the human body and there are no specific limitations to the applicable type of ultrasonic micro-bubble reagent. Fluorocarbon micro-bubble reagent may be used. To produce this kind of reagent, a human Albumin solution is processed with the aid of a sound vibration device. During this process, fluorocarbon gas is added to the Albumin solution at a steady rate. Medical salt-water base micro-bubble reagent can also be used. The process is similar to that mentioned above. Commercially available imported ultrasonic radiation micro-bubble reagent is also an option.

The reagent is injected into the area where the embolus needs to be formed and then irradiated with ultrasonic waves, forming the embolus. Experiments show that by applying low frequency ultrasonic radiation in combination with a micro-bubble reagent to animals with tumors, selective formation of embolus in blood vessels in tumors can be achieved in targeted areas. This invention provides a new and highly effective method to form blood vessel embolus and provides a new kind of medicine and way for blood vessel embolizing treatment of cancer.

The reagent is injected into the area where the embolus needs to be formed and then irradiated with supersonic waves and forms the embolus. There are not any specific requirements for the ultrasonic waves and generally low energy and low frequency ones are used. There are not any specific time requirements for treatment but 0.5 to 60 minutes is preferred. Studies show that low power ultrasonic radiation doesn't damage the tiny blood vessels. There also is not any damage when only micro-bubbles are injected without ultrasonic radiation. When micro-bubbles reagent is injected and ultrasonic radiation is applied, tiny blood vessels with diameters smaller than 7 μm appear damaged. Other research indicates that with micro-bubble reagent in the tissue, the quantity of cavitations nuclides is increased; it makes the cavitations effect occur with low power ultrasound which can be obtained by only high power ultrasonic radiation.

Irradiating the injected micro-bubble imaging reagent with low power ultrasonic radiation, creates a cavitations-effect in the tiny blood vessels in the tumors or the nearby tissues. The method breaks the walls of the tiny blood vessels and some of the nearby tissues. This method, activates the inner or outside source mechanical cruor, induces embolizing in the new blood vessels in tumors and interdicts the blood supply to the tumors in the treated area. Finally the tumor cells die and the tumor volume decreases and its growth is controlled. The purpose of treating cancer without pain in other normal tissue is accomplished.

This invention applies low power ultrasound to irradiate the injected micro-bubble imaging reagent through the vein to generate a cavitation effect, directly targets the blood vessel network or vasculature for supplying nutrition to the tumor. This invention can be used to embolize blood vessels in the tumors at different locations and at different stages. This invention makes Folkman's tumor treating theory “cutting blood supply and curbing the forming of new blood vessels in tumor” a reality. This new, pain-free cancer treatment method makes it possible to put new born blood vessels or neo-vasculature theory into clinical application. To this date, such a highly effective method has not been reported in China or other countries.

The ultrasonic radiation micro-bubble reagent used in this invention is a carbon dioxide (CO₂) type. This invention uses a large molecule substance as the carrier to wrap, stick, stabilize and carry the bubbles. Large molecule substances include all kinds of blood plasma substitutes, blood and plasma of the body, plasma of the same type, semi-lactose, glucose, lactose, Hetastarch, Human Serum Albumin, Dextran-70, Dextran-40, dextran-10, polygeline, Gelofusine, Polyvidone or Dxypolygelatin.

Semi-lactose and glucose have comparatively smaller molecular weights, lower viscosity and require a shorter period for stabilizing and carrying the bubbles.

Two methods are included in this invention for making the micro-bubble reagent:

1) Physically making the carbon dioxide gas micro-bubble reagent. Gas or liquid carbon dioxide is injected into solution with a large molecule substance.

2) Chemically making the micro-bubble reagent by reacting an Organic Acid such as Vitamin C with NaHCO₃ Vitamin C and NaHCO₃ can be injected into the human body as medicine, they react and carbon dioxide bubbles appear. Injecting this kind of micro-bubble reagent into the human body in combination with hollowing cavitations reagent to the targeted area. Irradiating this area with ultrasonic waves. The fat cells preserved can be destroyed.

Large molecule substances are used to guarantee the size and stability of the micro-bubbles. The large molecule substance serves as a carrier to wrap, stick, stabilize and carry the bubbles, particularly a plasma substitute type such as Hetastarch, etc.

There are numerous kinds of organic acids such as Vitamin C, lactic acid, citric acid, amber acid, tartar acid, lactose acid, semi lactose acid, glucose acid, amino glucose acid, amino acid, etc. For medical use, lactic acid, citric acid, glucose acid and amino acid are usually options.

Gas Type Carbon Dioxide Micro-Bubbles

A typical formulation is as follows:

100% Vitamin C (including above mentioned organic Acid) 25%

5% NaHCO₃ 50%

Hetastarch 25%

1 to 10 ml of reagent is injected per kilogram of body weight to produce micro-bubbles of up to 10⁶-10¹⁰ with diameter of 1-10 microns.

For clinical application, a maximum carbon dioxide tolerance shall be calculated based on the weight, height and surface area of the person involved, and adjusted accordingly within the above mentioned range.

Ultrasonic cavitations can result in tissue cavitations in a short period of time. The sound-cavity effect will occur so the cells will open to the surrounding large molecules and are crushed by high temperature and pressure. Under normal conditions, the cavitating nucleolus concentration is very low in the body fluid. Therefore high dose ultrasonic radiation is needed to obtain the cavitation effect, and this leads to damage of the nearby tissues while killing the targeted tissues. High dose ultrasound is not selective and of severe damage and can not be applied in the treatment of blood vessel embolizing. Research indicates, if micro-bubble reagent exists in the tissue, low dose ultrasonic radiation can create a sound cavitating effect as that otherwise can be obtained with high dose ultrasound radiation. The micro-bubble reagent applied in this invention is initially used for ultrasonic diagnosis. The reagent can reach the organs through the blood and increase the cavitating nucleolus rate in the targeted area.

In our experiment low frequency ultrasound radiation creates a cavitation, breaks the blood vessel walls and the surrounding tissues, activates cruor internally and externally, induces blood vessel thrombus embolus in a large area, and cuts blood supply to the targeted area. In the area where no micro-bubble reagent was present, few embolus were formed. We also conclude from the research that the embolizing rate was low where only ultrasonic radiation was used, only 34.15% of the blood vessels were embolized to various extents. When ultrasonic radiation was applied in combination with micro-bubble reagent, the embolizing rate increased significantly, up to 89.11%. In this invention, the reagent is injected to the area where the embolus needs to be formed, then irradiated with ultrasonic waves and the embolus is formed. Experiments have shown that by applying low frequency ultrasonic radiation in combination with a micro-bubble reagent to treat animals with tumors, selective formation of embolus in blood vessels in tumors can be obtained in the targeted area. This invention is a novel highly effective and damage-free method to form blood vessel embolus and provides a new kind of medicine and method for blood vessel embolizing treatment of cancer.

This invention applies the micro-bubble reagent which is the combination or mixture of an ultrasonic micro-bubble imaging substance and a tracing or marking isotope with targeted substance, so a y ray camera or SPECT equipment or Positron Electron Tracing (PET) equipment can be used to detect the isotope and locate the tumor precisely.

There are numerous kinds of isotope tracing or marking micro-bubble reagent with targeting substance, such as isotope marking albumin micro-bubbles. The isotope marking substance comprise ¹²⁵I, ¹²³I, ^{99m}Tc (^{99m}Tc-PYP, etc), ¹¹¹In, ¹¹C, ¹⁸F, ¹³N, ⁸²Rb, wherein the natural occurring pistron integration element is a positron integration radioactive nuclide such as ¹¹C, ¹³N, ¹⁵O, ¹⁸F can be applied in PET imaging, brain and cardiac muscle perfusion imaging, metabolizing imaging, benign and malignant tumor identification imaging.

The tracing or marking isotope with targeting substance which also has a treatment function includes: ³²P, ³⁵S, ¹⁹⁸Au, ^{99m}Tc (^{99m}Tc-PYP, etc.) ¹¹¹In, ¹²⁵I and ¹³¹I, ¹⁵³Sm—mainly EDTMP β injection treatment substance, ⁹⁰Y-GTMS, ⁸⁹SrCl₂, etc.

The targeting substance includes: Human Serum Albumin (^{99m}Tc-MAA), fluoral sodium, Colloid ^{113m}In, Marking Erythrocyte, EHIDA, ^{99m}Tc-MT, ¹³¹I-rose, Sulfer colloid, DTPA, EHIDA, ^{99m}Tc-DMSA, calcium gluconate, o-iodohippuric acid (OIHA). A better result of radioactive imaging and radiation treatment can be achieved by employing the targeting substance such as mono clone antibody of Molecule Nucleus Medicine, oncogene antisense oligonucleotide (asons) etc.

The micro-bubble reagent includes clinically applied fluorocarbon type, medical salt-water type, semi lactose bubble liquid, envelope bubble liquid, carbon dioxide generation type and large molecule weight substances that are used as the carrier of the ultrasonic reagent to wrap, stick, stabilize and carry the bubbles.

This invention's device comprises an ultrasonic micro-bubble imaging reagent injecting part, local positioning part and an ultrasonic treatment part. The injecting part injects ultrasonic micro-bubble imaging reagent as the embolizing reagent. The local positioning part serves to identify the area where the blood vessel embolus needs to be formed. The ultrasonic treatment part functions to irradiate the area locally with ultrasonic waves, therefore micro embolus can be formed in the targeted area and the blood vessels selectively embolized.

There are no specific requirements for the ultrasonic waves. Generally low energy and low frequency ultrasound are used so that the ultrasound will not cause any adverse effects to the normal human body. There are no specific requirements for treatment time, generally 0.5 to 60 minutes are used.

The device in this invention includes a hand-held ultrasonic treatment head with latex coupling and buffer protection device. The treatment head includes a metal treatment head, electrode patch, ceramic patch, range change rod, counter weight, power source, handler, terminals, plug, switch, signal lights and water film purse. There is a water purse on the treatment head. The mainframe of the medical power ultrasonic wave generator generates a power signal with a certain frequency. A piezoelectricity wafer in the treatment head converts electric power to vibration power, which is magnified through the swing change rod and then transferred to the treatment head. This invention uses water in the water purse to transfer the energy. The water purse can also provide coupling and buffer protection to the human tissues. The improved medical treatment head can be used in all applications including health care, treatment and hairdressing, particularly where the human body is involved.

After observing the formation of embolus in the tiny blood vessels of the rabbit liver by low power ultrasonic radiation and micro-bubble reagent, research on normal animal and animals with planted tumor were performed. We observed that the radiation of low power ultrasound on the injected micro-bubble reagent in the blood vessel induced a sound cavitating effect which damages the blood vessels and embolized, from which the tumor tissue dies or a large area of the tumor tissue becomes putrescence. While the normal liver and muscle tissues were not damaged. The damage to the tumor tissue caused solely by supersonic radiation is not apparent. We researched the combined use of low frequency ultrasound with a micro-bubble reagent to treat animals with tumors. Embolus were selectively formed in the blood vessels in the tumors. This method is a new and highly effective method to form blood vessel embolus and provide a new kind of medicine and way for blood vessel embolizing treatment of tumors. The system uses low power ultrasound to induce a micro-bubble reagent to form embolus in the blood vessels in the tumors. This invention meets the requirements of fighting tumors safely and effectively. This invention applies low power ultrasound together with micro-bubble reagent to form blood vessel embolus in tumors. This invention has advantages over surgery, chemotherapy, radiotherapy, thermotherapy, etc. The advantages are:
1. Good penetration for low frequency and low power ultrasonic radiation. The energy is low and generally only needs 0.5 W output power. No pain or tissue damage. It is precise, highly efficient and can treat shallow and deep tumors and is safe and does not have side effects.
2. Can externally treat tumors of different kinds, stages, sizes and positions from outside of the body.
3. Lower treatment cost, easy to operate and use.

This invention uses low power ultrasonic radiation to induce a micro-bubble reagent to form embolus in the blood vessels in tumors. This invention can be used together with radiotherapy and chemotherapy and provide auxiliary detection. This invention uses low power ultrasonic radiation to induce a micro-bubble reagent to form embolus in the blood vessels in tumors and to cut off the blood supply to the tumors. The ultrasonic treatment head in this invention is hand-held with a latex coupling and a buffer protection device to achieve the harmless ultrasonic transmission of power and is easy to use.

This invention researched patients with liver, kidney or other soft tissue tumors with the induction of ultrasonic-B or CT. The significant effect of ultrasonic radiation and micro-bubbles on the formation of embolus in blood vessels in the targeted tumors was observed.

The features of the micro-bubble reagent are:
1. Carbon dioxide type, easily dissolved in the human body and exhaled from the lungs, thus reducing the possibility of gaseous embolus.
2. Hetastarch colloid (plasma substitute) as the colloid to increase the stability of micro-bubbles in the blood, reduces the possibility of exhalation of carbon dioxide by the lungs and keep micro-bubbles for a longer period of time.
3. Hetastarch as the replacement of the human albumin to eliminate the danger of hypersusceptibility and contagious diseases caused by a blood contagion.
4. Similar to other micro-bubble reagents, can be injected into the artery or vein with similar result.
5. In case the quantity of micro-bubbles decreases, it can still be the cavitation nucleolus and, under ultrasonic radiation, cause a radiation effect, induce a sound cavity effect, destroy the walls of the micro and small blood vessels and leads to embolizing in small vessels and putrescence of the tissue.

This invention produces significant results. The micro-bubble reagent medicine is harmless and non-poisonous to the human body, the method does not cause pain and has no side effects. The curative effect is certain. This invention is applicable for malignant tumors at various stages. In addition to malignant lung tumors, tumors on the surface of the abdominal cavity, pelvic cavity and breast can be treated by this method. It is convenient and easily to be promoted. Due to its non-poisonous nature, this invention can also be used to treat benign tumors.

This invention combines a marking substance of an isotope with a targeting substance of human blood albumin to produce an ultrasonic micro-bubble imaging reagent. This invention completely overcome the following shortcomings: injected before treatment, targeting can not be achieved; but if after treatment, accurate targeting can not be achieved. In this invention radial y rays eradiated by isotope through SPECT can be used for tracing and detecting and radial β eradiated by isotope with strong ionization biological effect can be applied for close radioactive treatment of the tumors to kill tumor cells, monitor tumor cells at the same time and reinforce if necessary at any time. This invention will become a new method for resting therapy for treating tumors. The effectiveness assessment of this treatment can be done easily since the isotope marking substance does not have any way to re-enter or leave the tumor due to the embolus formed in the blood vessel. We can assess the single or combined effect of the treatment by the metabolizing scale.

Research of the biological effect of the isotope marking micro-bubble reagent is done through decorating the ultrasonic micro-bubbles to study their dynamic features; making of the isotope marking micro-bubbles and studying their metabolizing dynamics and distribution in an animal's body; developing know-how for ultrasonic radiation micro-bubble reagent; providing a scientific basis for clinical application; and at the same time providing a new method for treating cancer through embolizing. By applying ultrasonic micro-bubble reagent to create a sound cavity effect to embolize blood vessels, using isotope marking to achieve positioning and real time detecting and reinforcing the treatment timely, the treatment results can be observed; measurement forecasts can be made before and after and an isotope can be used as local radioactive treatment. All these provide a promising future for the treatment of cancer.

This invention is also very important to the development and application of diagnosis and treatment using ultrasonic micro-bubbles.

The research of ultrasonic radioactive micro-bubbles to form embolus in blood vessels in tumors, with the objective of embolizing induced by ultrasonic radiation, advances the radioactivity technology and benefits each technology. The use of isotope imaging resulted in precise targeting, damage free, real time detection and overcame the shortcomings of ultrasonic micro-bubbles. In the process of cancer treatment using isotope technology to achieve real time monitoring, timely treatment reinforcement and forecasting the future results.

This invention not only used low frequency and power ultrasound to induce the sound cavity effect to form embolus in blood vessels to treat cancer is applied, but also used the biological effect on tumor cells of ionization eradiating of radial: isotope dual treatments with one medicine.

This invention provides a new local treatment method of ultrasonic induced cancer treatment by using ^{99m}Tc, as the example of marking albumin micro-bubble. The main advantage of this local treatment is that it induces the overall toxicity of cancer treatment to the lowest level.

This invention involves physics, electronics, ultrasonic radiation, nucleus medicine and oncology. By applying their basic principles, this invention elaborates the mechanism of the harm-free treatment from various fields such as molecular pathology, molecular biology and so forth. This invention boosts the development of ultrasonic radiation, nucleus medicine and pathology and expands these subjects. This invention demonstrated the current trend of science and research: intercrossing, inter-penetrating, inter-supporting and inter-absorbing of multiple subjects. This invention makes the cancer treatment more scientific and practical.

### 4. EXPLANATIONS OF DRAWINGS

FIG. 1 Structure of the device of this invention.

FIG. 2 Photo comparison on embolizing of blood vessels in tumors.

FIG. 2A Single ultrasonic effect on normal liver tissue, no formation vessel embolus.

FIG. 2B Single ultrasonic effect on tumor tissue, no formation of vessel embolus.

FIG. 2C Combined effect of ultrasound and micro-bubble reagent on tumor tissue, formation of vessel embolus.

FIG. 3˜5 Comparison of tumor vessel embolus per this invention.

FIG. 3A Tumor tissue necrosis immediately after being treated only by ultrasonic radiation, no vessel embolus and tumor putrescence.

FIG. 3B Treated with ultrasound alone, one hour later, no vessel embolus and tumor putrescence.

FIG. 4A Treated with ultrasonic radiation and micro-bubble reagent, dead tumor tissue and vessel embolus and tumor putrescence illustrated.

FIG. 4B Treated with ultrasonic radiation and micro-bubble reagent, one hour later, vessel embolus and tumor necrosis shown.

FIG. 4C Treated with ultrasonic radiation and micro-bubble reagent, two hours later, vessel embolus and tumor necrosis shown.

FIG. 4D Treated with ultrasonic radiation and micro-bubble reagent, one day later, vessel embolus and tumor necrosis infarct illustrated.

The size of the above pictures: left: 10×110 in. right: 10×20 in.

FIG. 5 Treatment repeated with ultrasonic radiation+micro-bubbles reagent, (once a day, total 3 days) vessel embolus and tumor putrescence with bleeding shown. Picture size: left: 4×10 in. right: 10×10 in.

The ultrasonic micro-bubble reagent is injected as the reagent to form the embolus in the blood vessels, with the help of CT or B-ultrasound to identify the area which needs to be embolized, or selects the area by eye. Typically, the energy of the ultrasonic wave is transmitted from the surface of the body to the area where ultrasonic radiation+micro-bubble reagent has already been rejected, then the embolus will be formed in the blood vessels.

FIG. 6 Structure explanation of this invention

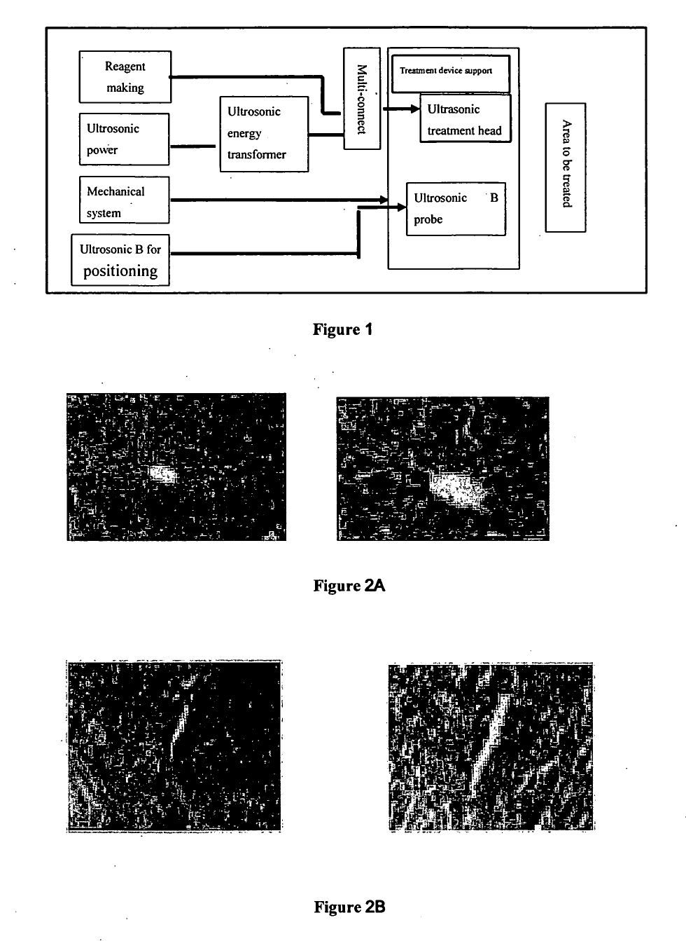

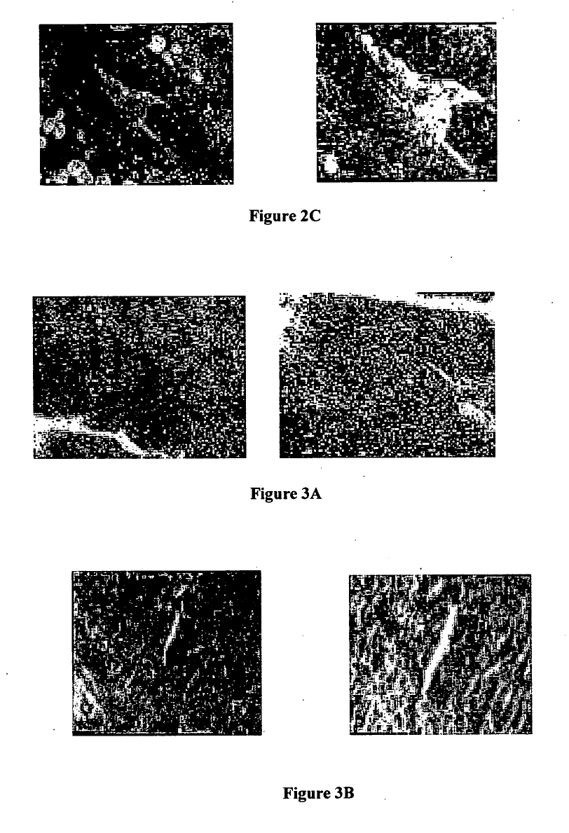

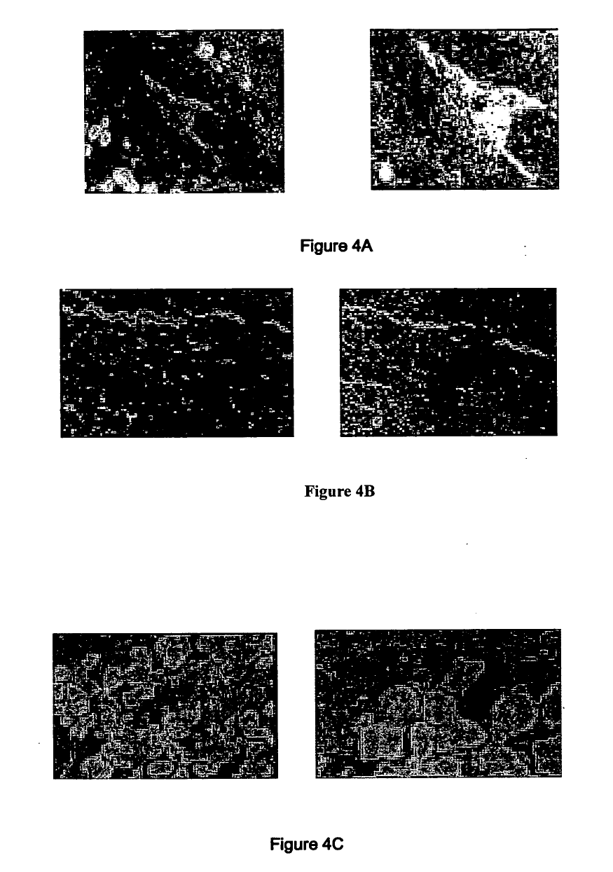

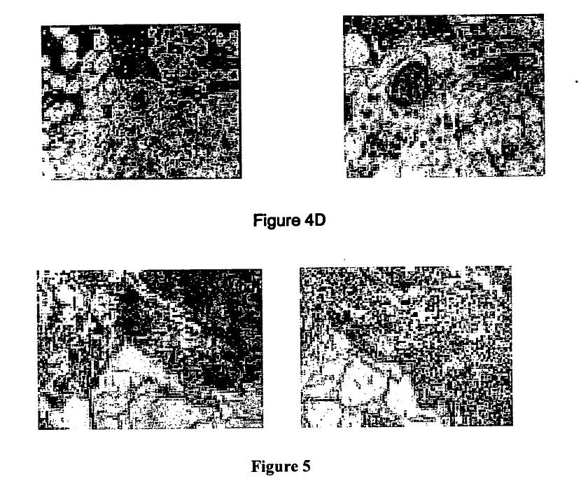

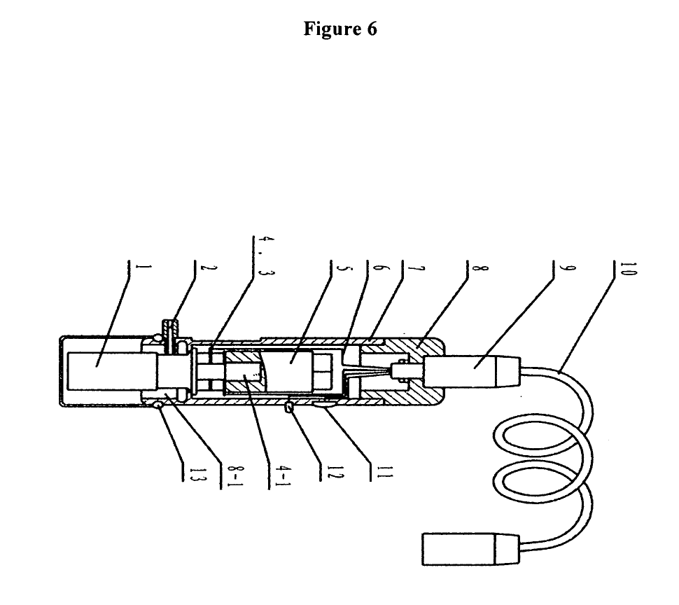

In FIG. 6, 1—metal treatment head, 2—connection, 3—electrode patch, 4—ceramic patch, 4-1—range change rod, 5—counter weight, 6—power source, 7—handler, 8,8-1—terminal, 9—plug, 10—power source, 11—switch, 12—signal light, 13—water purse.

### 5. EXAMPLES

A fluorocarbon micro-bubble reagent was used. It was prepared by taking 10 ml 5% human Albumin solution, putting it in a plastic injector, and being treated with a sound vibration device. During this treatment process, fluorocarbon gas was added to the Albumin solution at a steady rate. The diameter of the micro-bubbles reagent produced by this method was between 2.0˜5.0 μm, 98% of the bubbles' diameter were less than 10 μm; the concentration of the micro-bubbles was (1˜2)×10¹² per liter. The solution was injected into the vein at about 1-10 ml per kilogram of body weight. The injection amount is based on the area, the type of tumor treatment time to be treated.

The injection methods of ultrasonic micro-bubble reagent include: 1) venous injection; 2) artery injection; 3) artery and vein intubatton or retaining canula injection and 4) local injection.

The treatment period was from 0.5 to 60 minutes. For animal experiments, no significant difference was observed from treatment time, 2 minutes, 5 minutes, 20 minutes and 30 minutes.

The ultrasonic radiation micro-bubble reagents includes: fluorocarbon type, medical salt-water type and those mentioned below:

| | |
|---|---|
| 1) Albunex | (Molecular Biosystems Inc., USA) |
| 2) fso69 | (Molecular Biosystems Inc., USA) |
| 3) SHU454 | (Schering AG, Germany) |
| 4) SHU508 | (Schering AG, Germany) |
| 5) QW3600 | (Sonus Pharmaceuticals Cosla Mesa) |

Example for Using Carbon Dioxide Type Micro-Bubble Reagent

Solid Content Ratio Scope:

Vitamin C: NaHCO₃: Protein (plasma substitute)=10-35:1-3.5:20-80

Solvent was 3-10 times the solid content, particularly, concentration of NaHCO₃ was within 3-10%. The above ratios could be applied for citric acid, lactic acid and glucose acid, and amino acid should be much higher.

The optimal concentration of NaHCO₃ was 5%, better solid content ratio was:

Vitamin C or citric acid or lactic acid or glucose acid or amino acid: NaHCO₃: protein (plasma substitute)=20-30:2-3:40-60. Since Vitamin C or organic acids and plasma substitute may be injected into the human body alone, a strict ratio is not necessary. Excessive Vitamin C and NaHCO₃ has no harmful side effects on the human body, neither does plasma substitute. Of course, sufficient carbon dioxide may be produced through a proper mole ratio. This invention used a weight percentage ratio.

Vitamin C or other organic acid reacted with NaHCO₃ to produce carbon dioxide. Plasma substitute large molecule substance was the carrier of the ultrasonic reagent to wrap, stick, stabilize and carry the bubbles. The ratios of these three kinds of substance were:

| | | | |
|---|---|---|---|
| (1) 20:2:40 | (2) 30:3:60 | (3) 25:2.5:50 | (4) 20:2:60 |
| (5) 30:2:60 | (6) 30:3:40 | (7) 20:3:60 | (8) 10:2:20 |
| (9) 10:3.5:80 | (10) 10:3.5:80 | (11) 35:2:80 | (12) 35:3.5:70 |
| (13) 35:3:60 | (14) 35:2:70 | (15) 10:2.5:50 | |

The above ratios can also be applied for citric acid, lactic acid and amino acid.

There is no difference in using the following plasma substitutes registered with the Chinese State Pharmacopoeia:

| | |
|---|---|
| 1. Hetastarch | Chemical medicine category code: 401703060101 |
| 2. Human Serum | Chemical medicine category code: 4021070102115 |
| Albumin | |
| 3. Dextran-70 | Chemical medicine category code: 401703030501 |
| 4. Dextran-40 | Chemical medicine category code: 401703030401 |
| 5. Dextran-10 | Chemical medicine category code: 401703030201 |
| 6. Polygelatin | Chemical medicine category code: 401703090201 |
| 7. Gelofusine | Chemical medicine category code: 401703080101 |
| 8. Polyvidone | Chemical medicine category code: 401703050101 |
| 9. Dxypolygelatin | Chemical medicine category code: 401703090101 |

Additionally, the plasma substitutes also include: self body blood, self body plasma, same type plasma, semi lactose, glucose and lactose. The lactose and glucose mentioned above may be applied alone or as mixtures without differences.

An ultrasonic radiation micro-bubble reagent is injected as the reagent to form the embolus in the blood vessels. The CT or B-model ultrasonic is employed to identify the area which needs to be embolized and where the reagent should be injected. Typically, the energy of the ultrasonic waves is transmitted from the contacted surface of the body to the area where ultrasonic micro-bubbles reagent has already been injected, then embolus will be formed in the blood vessels. Another way is to inject a micro-bubble reagent to a precise area and selectively induce to form capillary embolus or destroy the cells.

Example of using amino acid: using medical or transfusionally applied cystine, lysine, glutamic acid, aspartate, phenylalanine, cysteine, etc. Refer to the above mentioned concrete ratios.

The Example is below (please refer to the related drawings)
1. A small white rat was the object of the experiment. Supersonic action alone could not embolize the blood vessels effectively.
2. Cardiac (artery) injection of micro-bubble reagent plus supersonic wave. The result was good.
3. Tail venous injection of micro-bubble reagent plus ultrasonic wave. No side effects and good treatment result.
4. When NaHCO₃ was used the concentration was 5% (weight-ratio)

The better range was Vitamin C:NaHCO₃:Plasma substitute=20-30:2-3:40-60. Solvent was injection grade water.

The example for producing gaseous carbon dioxide micro-bubble reagent physically entailed injecting medical carbon dioxide gas or liquid carbon dioxide into a solution of a large molecule substance under pressure. The large molecular substance includes various kinds of plasma substitute, self blood of the body, self plasma of the body, same type plasma, semi lactose, glucose, lactose, etc. Pressurized gaseous carbon dioxide micro-bubble reagent shall be kept in a pressurized tin. Shaking or vibration is forbidden before the tin is opened. After the tin is opened the reagent shall be put into use immediately to ensure the quantity and effectiveness of the micro-bubbles.

Example for Isotope Marking Micro-Bubble Reagent:

1. Making, Physical and Chemical Identification of the Micro-Bubbles.

1.1 Making of the Micro-Bubbles

Similar to the above micro-bubbles preparation, a 10 ml of 5%.g.ml⁻¹ human serum albumin with different sucrose concentration was prepared. The solution was then put into a 50 ml polytetrafluoroethylene plastic cup. The solution was then saturated with oxygen or perfluoropropane accordingly, then the head of the UGI type ultrasonic wave generator was placed just under the surface of the liquid. A 150 W power source was used to treat the solution for 1 minute at a fixed frequency of 20 Hz. The micro-bubbles so prepared were kept in a sealed container for testing.

Additionally, commercial product SHU508 semi lactose bubble liquid and florin-carbon micro-bubble reagent Albunex could also be applied.

As for the production of fluoro carbon imaging reagent, take 10 ml 5% human Albumin solution, put it into a plastic injector and then treated with a sound vibration device. During this treatment process, fluorocarbon gas was added to the Albumin solution at a steady speed. The diameter of the micro-bubbles reagent prepared from this method was between 2.0˜5.0 μm, with 98% of the bubbles' diameter less than 10 μm. The concentration of the micro-bubbles was (1˜2)×10¹² per liter. The injection method of ultrasonic micro-bubble reagent included: 1) artery injection; 2) venous injection; 3) artery and vein intubatton or retaining canula injection and 4) local injection.

Meanwhile, carbon dioxide type micro-bubble reagent was prepared. Vitamin C reacted with NaHCO₃ to produce carbon dioxide. Hetastarch acted as the carrier of the ultrasonic reagent to wrap, stick, stabilize and carry the bubbles. The size of the carbon dioxide type bubbles is bigger, about 20 μm in diameter.

In order to facilitate the integration with ^{99m}Tc nuclide, the pH of the solution should be 6. For different nuclides, the pH should be different.

1.2. Testing the Performance of the Bubbles

The micro-bubbles were tested separately 1 hour and 24 hours after it they were prepared. Heat resistant performance testing was completed by the survival rate of the micro-bubbles at 5 different temperature points. The time interval for testing was 30 minutes. A constant temperature procedure was obtained by a constant temperature water bath. The concentration of the micro-bubbles was measured by a cell counting device and microscope. Micro-bubbles were separated from the surroundings by adjusting the color-comparison glass of the microscope. The size of the micro-bubbles could be estimated by the gauge on the microscope. The video image was exported to a computer by the camera mounted on the microscope. The performance of the syntonic wave was tested by an ultrasonic device. A small quantity of milk was used as the background diffraction source. The reflection signal of the metal sheet was compared with that of the imaging agent.

The pH was 6. The performance test of the micro-bubbles indicated that the survival rate of the micro-bubbles was more than 90% within an hour for the above examples. They all could be put into clinical application.

2. Making of the Isotope Marking Micro-Bubbles

2.1 Making of Semi-Product

1. Put 4 ml of 25% human serum albumin into 2 ml SnCl.2H₂O solution (concentration of 1 mg/ml).

2. Use 1N NaOH to adjust the pH.

3. Use medical salt-water to dilute the human serum albumin to no less than 135 mg/ml.

2.2 Marking

Put 0.1-1 ml disinfected Na^{99m}TcO₄ into 1 ml of the above semi-product solution, then mix for 1 minute to be ready for clinical use.

Making of albumin ^{99m}Tc: freeze-dry 2.1 mg of albumin, 126 μg SnCl₂2H₂O and a small quantity of benzyl alcohol in an ampoule, add ^{99m}TcO₄ and mix well to form a yellow color liquid suspension, keep for 5 minutes under room temperature, and it was mix again for a few seconds before injecting into the vein. An ultrasonic wave detecting head may be used in this kind of viscous albumin solution or gel solution to produce micro-bubbles.

The shape, size and size distribution of the Albumin ^{99m}Tc micro-bubbles had no significant difference with that of the regular micro-bubbles. The range of the micro-bubbles' size was 20-50 μm.

^{99m}Tc micro-bubbles can be kept stable for a period of time. ^{99m}Tc micro-bubbles combined with monoclonal antibody may be produced based on its introduction. Injection grade isotope nuclide such as MIBI or ¹³¹I or ¹²⁵I-o-iodohippuric acid (OIHA) injection reagent can be directly applied.

Isotope marking albumin micro-bubbles was injected into the vein in the tail of a Wister rat and the radioactivity of each organ was tested at different times, then the data was processed in a computer and the medical dynamic parameter was obtained.

Distribution of Isotope Marking Micro-Bubbles in the Big Rat's Body

Isotope marking albumin micro-bubbles were injected through the vein of the rat. The radioactivity of the blood, heart, liver, kidney, spleen, brain, lung, bone were tested at 2 minutes, 30 minutes, 60 minutes, 120 minutes after the injection.

Radioactivity Self-Imaging of the Rat Body

After injected an isotope marking albumin micro-bubbles into the vein in the tail of the normal Wister rat, the rat's body was dipped immediately into the mixture of acetone and dry ice at −80° C.; then buried in a substance made from 8% hydroxymethyl-cellulose and refrigerated for 2 hours at −80° C.; and then sliced up with an LKB-2250PMV slicing machine. The thickness of the slices was 40 μm. After the rat's body was dehydrated and dried, it was scanned with a GS-250 molecule imaging system and the radioactivity distribution of the isotope marking albumin micro-bubbles in the rat's body and brain was observed.

Biological Effect of Ultrasound Induced Blood Vessel Embolus with Isotope ^{99m}Tc Marking Micro-Bubble Reagent

Twelve rats were chosen for the control group and 12 rats for the experimental group, micro-bubbles were injected through the vein for the control group and isotope marking albumin micro-bubbles were injected into the vein for the experimental group. After the injection, the pathology of the heart, liver, kidney, spleen, brain, lung and bone were observed at 2, 30, 60, 120 minutes, 24 hours, and 48 hours by microscope. At the same time an isotope imaging detection proceeded. The result showed that the radioactivity was distributed evenly. For treating liver cancer, the monoclonal antibody of anti oxidation protein recipient was used on the 12 rats with liver cancer in the experimental group.

The biological effect of the blood vessel embolizing of the isotope marking micro-bubbles on the rats with liver cancer was observed. The radioactive concentration in the enriched liver. After 5 to 10 days the tumor inhibition was more pronounced in rats in the control group. An immunological organic chemistry method and the original bit hybridizing of molecule pathology method were used to detect the blood vessel damage of the tissue sample of the normal animals in the experiment group than animals planted with tumors. Radioactive nuclide liver blood vessel imaging was applied to observe the blood flow change in the radiated area. A positive result was obtained.

¹³¹I had the similar effect.

3. The Biological Effect of Applying Low Frequency, Low Power Ultrasonic Radiation on Isotope (¹³¹I, ¹²⁵I) Marking Albumin Micro-Bubbles to Induce Embolizing of the Blood Vessel of the Rat with Liver Cancer

Twelve rats were chosen for the control group and 12 rats for the experimental group, ^{99m}Tc marking albumin micro-bubbles was injected through the vein for the control group, and ¹³¹I isotope marking albumin micro-bubbles for the experimental group. Low frequency and low power ultrasound was applied to induce embolizing of the local blood vessels. The pathology of the heart, liver, kidney, spleen, brain, lung and bone was observed 2, 30, 60, 120 minutes, 24 hours, and 48 hours after the injection. At the same time, isotope imaging detection proceeded. The effect was the same as mentioned above.

The embolizing rate was up to 90% when ultrasound and micro-bubble reagents were used. The addition of isotope ^{99m}Tc did not significantly increase the embolizing rate.

The output power of the ultrasonic energy transforming device was 1-100 W, typically 5-30 W. The frequency was 20-50 kHz. Under this condition, the ultrasonic radiation does not cause any harmful side effects to a normal body. Various ultrasonic radiation micro-bubble imaging reagent may be medically applied in this invention. The handling time period was a wide range, from 0.5-60 minutes.

4. Carbon Dioxide Type Micro-Bubble Reagent was Applied as Ultrasonic Micro-Bubble Contrast Reagent and a Large Molecule Substance was Used as the Carrier to Wrap, Stick, Stabilize and Carry the Bubbles.

The typical formulation was:

Vitamin C (Vitamin C, including the above mentioned various organic acid) 25% (concentration 100%)

NaHCO₃ 50% (concentration 5%)

Hetastarch 25%

1 to 10 liters of the reagent per kilogram of the body weight was injected. The quantity of micro-bubbles produced was up to 10⁶-10¹⁰, with a diameter between 1-10 microns.

For clinical application, the maximal carbon dioxide tolerance level should be calculated based on the weight, height and surface area of the person involved to be adjusted within the above mentioned range.

The carbon dioxide type micro-bubble reagent can be dissolved easily into the body and exhaled from the lungs so it may reduce the possibility of micro-bubbles forming gaseous embolizing.

Hetastarch (plasma substitute) colloid may improve the stability of micro-bubbles in the blood and decrease the exhaling rate of carbon dioxide from the lungs so the micro-bubbles may be kept longer.

By using Hetastarch to replace Human Serum Albumin (such as in fluorocarbon human serum albumin), the danger of allergies and blood borne contagious diseases may be eliminated. ^{99m}Tc generator and ^{99m}Tc from Beijing Atomic High-tech Nuclear Technology Holding company may be used to replace other product.

5. Routine Method May be Applied for Radioactive Isotope Marking in this Invention
5.1 Isotope Changeover Method. It is the simplest way to make the marking compound. Under specific conditions, mix common compound Ax to be marked with radioactive compound Bx*, radioactive nuclide X of the radioactive compound may be exchanged with non-radioactive isotope X of the common compound, so AX*, AX+BX* and AX*+BX can be obtained.
5.2 Chemosynthesis Method. This method is commonly used for making the radioactive nuclide marking compound. Taking the simplest radioactive compound as raw material, make various marking compounds based on the principles of chemical synthesis. An intermediate may also be applied to make the synthetic process simpler.
5.3 Single Nuclide Marking Method. Some organic compounds, like protein and polypeptide can be marked with an isotope through a simple chemical reaction. For instance, the preparations of ¹³¹I Iodid Protein. The proteins, polypeptides or some non protein compounds can be marked by iodine as long as they are connected with tyrosine molecule. The most commonly used marking methods are Chloramine-T method and Lodogen method. Na¹³¹I is first oxidized to ¹³¹I₂, ¹³¹I₂ can then replace the two hydrogen atoms on the hydroxy bit of tyrosine-aroma-band through metathesis reaction, so radioactive iodine marking protein is obtained.
5.4 Complex Compound Making Method. It is an important branch of Chemosynthesis and a commonly used method for making marking compounds in Nuc-medicine, 80% of the current marking compound is obtained through this method. The reason is that ^{99m}TC and ^{113m}In generators are widely applied and it is of significant importance in Nuc-medicine. Through a complexation with metal ions by coordinate bond, dozens of marking compounds can be obtained for ^{99m}Tc alone.
5.5 Biological synthesis method. Through physiological and metabolizing process the method introduces simple radioactive nuclide marking compound into plant, animal and microorganism. Some complicated marking compounds that can not be obtained through chemical synthesis are produced by this method, such as proteins, hormones, etc.

For instance, a patient who weighs 75 kg is given 10 milli-courier of ³²P. Assuming that all ³²P is absorbed completely and distributed evenly in the body and no excretion. What is the total absorption of the tissues? The radioactive concentration of ³²P is evenly distributed in the body. This invention follows this method when the treatment used marking or tracing isotope with a targeting substance.

Implementing Example of the Device

The device in this invention is existing technology. A better result can be achieved by low energy and low frequency ultrasound. The frequency is about 20-50 kHz and the output power of the ultrasonic energy transforming device about 0.5-100 W. Such energy will not cause any harmful side effects to the normal body. The treatment time is 0.5-60 minutes. A treatment time of 20 minutes and 30 minutes did not have a significant effect on the result in the animal trials.

Structure of Injecting Device for Ultrasonic Micro-Bubble Imaging Reagent

The injecting device includes an injector and a vibrating device. The diameter of micro-bubbles produced by this device is between 2.0˜5.0 μm, with 98% of the bubbles' diameter less than 10 μm; the concentration of the micro-bubbles is (1˜2)×10¹² per liter. The reagent is injected into the vein. This invention first applies B ultrasound to determine the area to be treated; injecting the micro-bubble reagent into the peripheral blood vessels or to the area to be treated through intubatton; and then apply low frequency and low energy ultrasonic radiation to irradiate this area. The ultrasonic micro-bubble imaging reagent forms embolus in blood vessels. As for a typical area with tumors, the energy of ultrasonic waves will be transmitted from the surface of the body to the area where ultrasonic micro-bubble imaging reagent exists so embolus will be formed in the blood vessels.

Example for Treatment Head

The ultrasonic metal treatment head protruded out of the terminal, and the water purse or water film purse (13) covered and connected to the terminal. On the terminal, there is a drainage connector (2) to drain excessive water and ensured water in the purse was full. After water was drained through the drainage connector (2), a plug was used to plug the drainage connector. The water film purse (13) was preferably made of latex and can be replaced. Other liquid may also be used in the purse for effective ultrasonic coupling.

1-14. (canceled)

15. A method for embolizing blood vessels, comprising:
injecting an ultrasonic radiation micro-bubble reagent into a blood vessel; and
irradiating said reagent with an ultrasonic wave in an area where embolus are desired to be formed.

16. The method of claim 15, wherein the irradiation causes said reagent to form blood vessel embolus in said area.

17. The method of claim 15, wherein the ultrasonic radiation is of a low frequency and a low energy.

18. The method of claim 17, wherein the low frequency ranges from about 20 to about 50 kHz and the low energy comprises a power of about 0.3 to about 3 W.

19. The method of claim 15, wherein the irradiation is performed for about 0.5 to about 60 minutes.

20. The method of claim 15, wherein the reagent is injected at a dose of about 1 to about 10 ml/kg.

21. A method for reducing the size of a tumor, the method comprising:
providing an ultrasonic radiation micro-bubble reagent in a vessel providing blood to the tumor; and
irradiating said reagent with ultrasonic radiation to cause said reagent to form blood vessel embolus.

22. The method of claim 21, wherein the reagent is injected at a dose of about 1 to about 10 ml/kg and the ultrasonic radiation is of a low frequency and a low energy.

23. A method for treating cancer, the method comprising:
providing an ultrasonic radiation micro-bubble reagent in a vessel providing blood to a tumor of the cancer; and
irradiating said reagent with ultrasonic radiation to cause said reagent to form blood vessel embolus.

24. The method of claim 23, wherein the reagent is injected at a dose of about 1 to about 10 ml/kg and the ultrasonic radiation is of a low frequency and a low energy.

25. A ultrasonic radiation micro-bubble reagent for causing embolus, the reagent comprising Albunex, Optison, fluorocarbon micro-bubble reagent, medical salt-water base micro-bubble reagent, fso69, SHU454, SHU508, QW3600, a carbon-dioxide based reagent, or combinations thereof.

26. The reagent of claim 25, the carbon-dioxide based reagent containing a large molecule substance as a carrier for the reagent.

27. The reagent of claim 26, wherein the large molecule substance comprises blood, plasma, substitute blood, substitute plasma, Semi-lactose, Glucose, Lactose, Hetastarch, Human Serum Albumin, Dextran-70, Dextran-40, Dextran-10, Polygeline, Gelofusine, Poly-vidone or Dxypolygelatin.

28. The reagent of claim 26, wherein the carbon-dioxide gaseous based reagent is produced by adding carbon dioxide gas or liquid into a solution with the large molecule substance under pressure.

29. The reagent of claim 26, wherein the carbon-dioxide gaseous based reagent is produced by reacting an organic acid and NaHCO₃.

30. The reagent of claim 29, wherein the organic acid comprises vitamin C, lactic acid, citric acid, amber acid, tartar acid, lactose acid, semi-lactose acid, glucose acid, amino glucose acid, amino acid, or combinations thereof.

31. The reagent of claim 25, further comprising a marking or tracing isotope combined with a targeting substance.

32. The reagent of claim 31, wherein the combined marking or tracing isotope with a targeting substance comprises: ¹²⁵I, ¹²³I, ^{99m}Tc (^{99m}Tc-PYP, etc), ¹¹¹In, ¹¹C, ¹⁸F, ¹³N, and ⁸²Rb, wherein the natural occurring positron integration element is a positron integration radioactive nuclide such as ¹¹C, ¹³N, ¹⁵O, ¹⁸F; ³²P, ³⁵S, ¹⁹⁸Au, ^{99m}Tc, (^{99m}Tc-PYP, etc . . . ), ¹¹¹In, ¹²⁵I, ¹³¹I, ¹⁵³Sm-EDTMP β-injection treatment substance, ⁹⁰Y-GTMS, ⁸⁹SrCl₂, or a combination thereof.

33. The reagent of claim 31, wherein the targeting substance combined with the isotope includes: Human Serum Albumin (^{99m}Tc-MAA), floral sodium, Colloid^{113m}In, Marking Erythrocyte, EHIDA, ^{99m}Tc-PMT, ¹³¹I-rose, DTPA, EHIDA, ^{99m}Tc-DMSA, calcium gluconate, O-iodohippuric acid, molecular nucleus medical sole clone antibody, oncogene antisense oligonucleotides, or combinations thereof.

34. A medical device for forming capillary vessel embolus, comprising:
an embolizing agent injecting portion;
a positioning portion; and
an ultrasonic treating portion.

35. The device of claim 34, wherein the embolizing agent injecting portion injects an ultrasonic micro-bubble imaging reagent.

36. The device of claim 34, wherein the positioning portion positions the device to the location where capillary blood vessel embolus needs to be formed.

37. The device of claim 34, wherein the ultrasonic treatment portion comprises an ultrasonic energy output head with an energy and output frequency of about 20 to about 50 kHz and an output power of about 1 to about 100 W.

38. The device of claim 36, wherein the local positioning portion is an B Ultrasonic or X-Ray, CT or ECT.

39. An ultrasonic treatment head for a device for forming capillary vessel embolus, the head comprising:
a metal treating head;
an electrode patch;
a ceramic patch;
a range change rod;
a counter weight;
a power route;
a handler;
a terminal; and
a covered water purse.

40. The treatment head of claim 39, wherein it is hand-held apparatus

41. The treatment head of claim 39, wherein said head protrudes from the terminal, the water purse covers the terminal, and the water purse is made of latex.

42. The treatment head of claim 39, further comprising a drainage connection on the terminal.
